(19) United States
(12) Patent Application Publication
(10) Pub. No.: US 2006/0178696 A1
(43) Pub. Date: Aug. 10, 2006

(54) Macroporous materials for use in aneurysms

(76) Inventors: Stephen Christopher Porter, Oakland, CA (US); Victoria E. Carr-Brendel, Pleasanton, CA (US)

Correspondence Address: ROBINS & PASTERNAK, 1731 EMBARCADERO ROAD, SUITE 230, PALO ALTO, CA, 94303, US

(21) Appl. No.: 11/051,578

(22) Filed: Feb. 4, 2005

(51) Int. Cl. A61M 29/00

(52) U.S. Cl. 606/200

(57) ABSTRACT

This is a device for occluding a space within the body. In particular, the device comprises a porous material having a pore size of greater than about 30 microns and in which most of the pores of the porous material are co-continuous. The devices may be placed in a desired site within a mammal and are useful in inhibiting the formation of scar tissue.

### FIELD OF THE INVENTION

Compositions and methods for repair of aneurysms are described. In particular, macroporous materials that enhance healing in the aneurysm are disclosed, as are methods of making and using these devices.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings which may be dimensioned to engage the walls of the vessels. (See, e.g., U.S. Pat. No. 4,994,069 to Ritchart et al.) Other less stiff helically coiled devices have been described, as well as those involving woven braids. See, e.g., U.S. Pat. No. 6,299,627.

U.S. Pat. No. 5,354,295 and its parent, U.S. Pat. No. 5,122,136, both to Guglielmi et al., describe an electrolytically detachable embolic device. Vaso-occlusive coils having little or no inherent secondary shape have also been described. For instance, co-owned U.S. Pat. Nos. 5,690,666; 5,826,587; and 6,458,119 by Berenstein et al., describes coils having little or no shape after introduction into the vascular space. U.S. Pat. No. 5,382,259 describes non-expanding braids covering a primary coil structure.

Vaso-occlusive devices comprising one or more coatings have also been described. U.S. Pat. No. 6,280,457 discloses vaso-occlusive devices that include biodegradable coatings. U.S. Pat. No. 6,602,261 describes vaso-occlusive devices comprising an elongate flexible carrier and hydrogel materials having a porosity less than 25 microns. U.S. Pat. No. 6,245,090 describes vaso-occlusive devices comprising foam polymer materials having a porosity less than 250 microns with an open cell structure and including a radioopaque material. U.S. Pat. No. 5,456,693 describes vaso-occlusive devices comprising a collagen plug having a porosity greater than 50 microns.

Thus, none of the above documents show implantable devices as described herein including one or more macroporous materials in combination with a structural element, where the macroporous materials that limit the formation of scar tissue and resist long-term recanalization of an aneurysm.

### SUMMARY OF THE INVENTION

Thus, this invention includes novel occlusive compositions as well as methods of using and making these compositions.

In one aspect, the invention comprises a vaso-occlusive device comprising a macroporous material having a nominal pore size greater than about 30 microns, wherein at least 50 percent of the pores are interconnected with an adjacent pore. In certain embodiments, the pore size is between about 40 microns and about 400 microns. In other embodiments, at least 80% of the pores are interconnected with an adjacent pore.

In any of the devices described herein, the macroporous material may comprise a polymer, for example silicones, polytetrafluoroethylene, polyesters, polyurethanes, proteins, hydrogel materials and/or combinations thereof.

In another aspect, any of the vaso-occlusive devices described herein may further comprise one or more structural elements. In certain embodiments, the macroporous material at least partially surrounds the structural element(s). In other embodiments, the macroporous material is at least partially surrounded by, the structural element(s). In still further embodiments, the macroporous material at least partially surrounds and is at least partially surrounded by the structural element(s).

In other aspects, any of the devices described herein may further comprise two or more additional members (e.g., structural elements such as coiled or braided member). In certain embodiments, the additional member(s) is itself a vaso-occlusive device. The macroporous material may surround and/or be surrounded by the additional structural element(s). In certain embodiments, the macroporous material is attached to the structural member(s) at one or more locations. In any of the devices described herein, the additional member(s) may comprise a metal (e.g., nickel, titanium, platinum, gold, tungsten, iridium and alloys or combinations thereof), stainless steel or a super-elastic metal alloy. The structural element(s) may be, for example, be shaped as a helical coil. In any of the devices described herein, the structural element(s)may further comprise a biodegradable material and/or a bioactive component.

Any of the devices described herein may further comprise a severable junction detachably which may be connected to a pusher element. The detachment junction can be positioned anywhere on the device, for example at one or both ends of the device. In certain embodiments, the severable junction(s) are, an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof. The detachment junction(s) may be attached to macroporous material or one or more additional vaso-occlusive members.

In another aspect, a method of occluding a body cavity is described, the method comprising introducing any of the implantable devices as described herein into the body cavity. In certain embodiments, the body cavity is an aneurysm.

These and other embodiments of the subject invention will readily occur to those of skill in the art in light of the disclosure herein.

### BRIEF DESCRIPTION OF THE FIGURES

FIG. 1 is a perspective view of an exemplary macroporous material as described herein for use in promoting wound healing in an aneurysm, typically in combination with a structural element (e.g., vaso-occlusive coil).

FIG. 2 is a partial side-view, partial cross-section view of an exemplary device comprising an implantable macroporous material as described herein surrounding a coil-shaped vaso-occlusive device.

FIG. 3 is a partial side-view, partial cross-section view of an exemplary device according to FIG. 1. Within the deployment catheter, the macroporous material is shown in a compressed configuration.

FIG. 4 is a partial side-view, partial cross-section view of an exemplary device comprising an implantable macroporous material as described herein in combination with a tubular braided covering.

FIG. 5 is a partial side-view, partial cross-section view of an exemplary device comprising an implantable macroporous material as described herein in combination with a coil shaped outer covering.

It is to be understood that the drawing depicts only an exemplary embodiment and is not to be considered limiting in scope.

### DESCRIPTION OF THE INVENTION

Occlusive (e.g., embolic) compositions are described. The implantable macroporous biomaterials described herein have an appropriate architecture that promotes new vessel formation and maintains healthy viable tissue within and around the implant. Methods of making and using these vaso-occlusive elements also form aspects of this invention.

All documents (publications, patents and patent applications) cited herein, whether above or below, are hereby incorporated by reference in their entireties.

It must be noted that, as used in this specification and the appended claims, the singular forms “a”, “an”, and “the” include plural references unless the content clearly dictates otherwise. Thus, for example, reference to an implant comprising “a channel” includes implants comprising of two or more of such elements.

The implantable devices described herein comprise a macroporous biomaterial that is space-filling within an aneurysm and promotes long term, persistent foreign body responses to the material that does not become scar tissue. By “macroporous” is meant that a material that having a porosity of greater than 40 microns, generally between about 40 and about 400 microns (or any value therebetween), for example from about 40 to about 100 microns (or any value therebetween), from about 100 to about 200 microns (or any value therebetween), from about 200 microns to about 300 microns (or any value therebetween), or from about 300 to about 400 microns (or any value therebetween). Generally, the pores of the macroporous material are amorphous in shape. Preferably, some or all of the pores are interconnected.

By “biomaterial” is meant any substance or combination of substances synthetic or natural in origin, which can be used for any period of time, as a whole or as a part of a system that treats, augments, or replaces any tissue, organ, or function in a subject (e.g., mammal).

Aneurysms treated with known vaso-occlusive devices may result in the formation of scar tissue over time. Because scar tissue is an avascular, acellular mass made up mostly of extracellular matrix proteins, the interface between the scar tissue and healthy tissue is less robust than surrounding tissue and, as such, less resistant to long-term recanalization. The vaso-occlusive devices described herein comprise materials that are capable of arresting wound healing in a state of a modified foreign body response (also referred to as material microarchitecture-driven neovascularization) so that the resulting tissue response does not evolve into scar and, as such, is adjacent (continuous) with the healthy tissue and is more resistant to sheer, flow and recanalization. Notably, there is no demarcation between the response to the foreign body and the native tissue—they are co-continuous.

During a typical course of wound healing, neutrophils are the predominant cell type at the site of injury within the first 24-48 hours, killing and phagocytosing bacteria and/or cellular debris. After approximately 48 hours, macrophages become the predominant cell type, further removing cellular and foreign debris from the wounded area. Within three to four days, fibroblasts migrate out of the surrounding connective tissue (e.g., intima) into the wound area and begin to synthesize collagen, which quickly fills the wound space, forming a complex tertiary structure consisting of both cells and extracellular matrix components. New blood vessels also begin to grow into the area at this time to supply oxygen and nutrients needed by the metabolically active fibroblasts and macrophages. However, in a typical course of wound healing, new vessel formation begins to regress in the second week, resulting formation of an avascular and acellular scar.

Macroporous biomaterials as described herein provide tissue biomaterial anchoring and promote in-growth throughout the pores. The resulting “hallway” or “channel” pattern of tissue growth are healthy, space-filling masses that persist over the duration of the implant and promote host cell integration between the aneurysm wall and implant and throughout the aneurysm space. Most or all of the pores of the biomaterials described herein are preferably interconnected (co-continuous). The co-continuous pore structure of the biomaterials promotes space-filling in-growth of cells between aneurysm wall and implanted material. Thus, the macroporous implants described herein promotes appropriate wound healing within the aneurysm environment and throughout the porous material. Preferably at least 50% of the pores have interconnections with adjacent pores. More preferably the inter connectivity is above at least about 80%. Additionally, the material is mostly void space, consisting of a lattice work that cells grow between and around. Although other examples may exist where macroporous material is used within an aneurysm, the materials do not specifically call out the need for the host cells to in-grow and become co-continuous with other host cell-filled pores. See, e.g., International Patent Publications WO 04/078023; WO 04/103208; WO 04/062531; and WO 04/037318. Other technologies may allow for protein adsorption or absorption, but again do not promote cellular in-growth throughout via an interconnected macroporous structure. Thus, unlike other described macroporous biomaterials, the co-continuous pore structure of the materials described herein promotes host cell in-growth with concomitant neovascularization, and, in addition, that enhances cell and vessel persistence within the pores.

An additional advantage of certain types of macroporous materials (elastomers, for example) is that the air can be forced out of the pores (the material can be compressed) until the material is delivered, and then the material can relax back to its preferred (native) state. This offers the advantage of delivering the material through smaller diameter catheters than the final, non-compressed material.

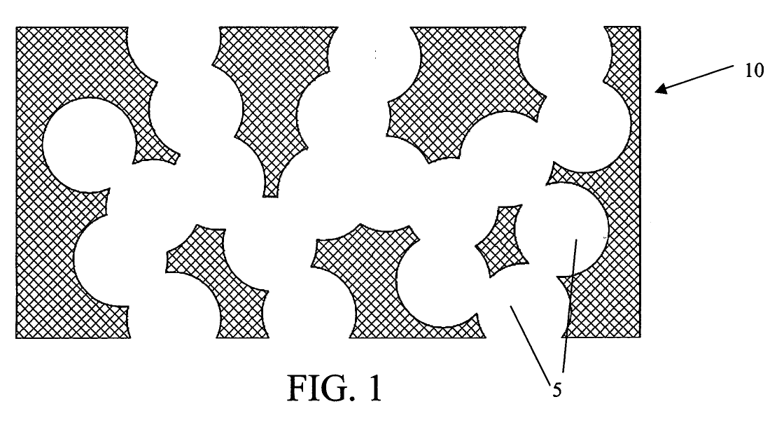

FIG. 1 depicts an exemplary embodiment of the inventive macroporous implants described herein. The device as a whole is generally designated (10) and is shown in a three-dimensional block. FIG. 1 shows an embodiment in which all of the pores (5) are interconnected.

The macroporous implants as described herein preferably comprise one or more materials that favor an arrested foreign body response, which as described above is granular in nature, has new vessel formation. Typically, the macroporous material comprises one or more materials having an average pore size ranging from approximately 20 microns to about 400 microns (or any value therebetween), more preferably from about 30 microns to about 300 microns (or any value therebetween), and even more preferably from 40 microns to about 200 microns (or any value therebetween), using conventional methods for determination of pore size (porosity) in the trade.

Non-limiting examples of suitable macroporous materials include natural and synthetic materials such as silicone, ePTFE, polyurethane, collagen and/or hydrogels. Methods for introducing suitable porosity in these materials are well known and include methods described in U.S. Pat. No. 4,076,656; U.S. Pat. No. 5,681,572; U.S. Pat. No. 6,602,261 as well as International Patent Publications WO 04/078023; WO 04/103208; WO 04/062531; and WO 04/037318. Although the use of some of these macroporous materials have been used as scaffolds to promote granular tissue in-growth, (see, e.g., U.S. Pat. No. 6,713,079, Seare et al (1993) ASAIO Journal 39: M668-M674) has been described, they have not been used as vaso-occlusive devices, likely because of their known anti-thrombogenic characteristics or lack of delivery systems to the vasculature.

Furthermore, although hydrogel and other materials have been proposed for use in aneurysm repair (see, e.g., U.S. Pat. Nos. 6,818,018 and 6,602,261), these hydrogels are not macroporous, having a porosity of less than 25 micron. Additionally, some hydrogels are incompatible with large pore architectures as gels may lack the strength to be deployed and maintain association between pores (e.g., they fracture or break apart. Furthermore, unlike previously described foam polymer devices (see, e.g., U.S. Pat. Nos. 6,245,090 and 5,456,693), at least about 50% of the pores of the macroporous elements described herein are interconnected. As described herein, interconnectedness between the pores induces the type of persistent granular tissue that will result in durable aneurysm treatment.

The macroporous materials of the devices described herein may include one or more fibers, strands, coils, globules, cones or rods of amorphous or uniform geometry that are smooth or rough.

The macroporous devices described herein can also be optionally used in combination with other vaso-occlusive members, for example the GDC-type vaso-occlusive coils described above (see, e.g., U.S. Pat. Nos. 6,723,112; 6,663,607; 6,602,269; 6,544,163; 6,287,318; 6,280,457 and 5,749,894).

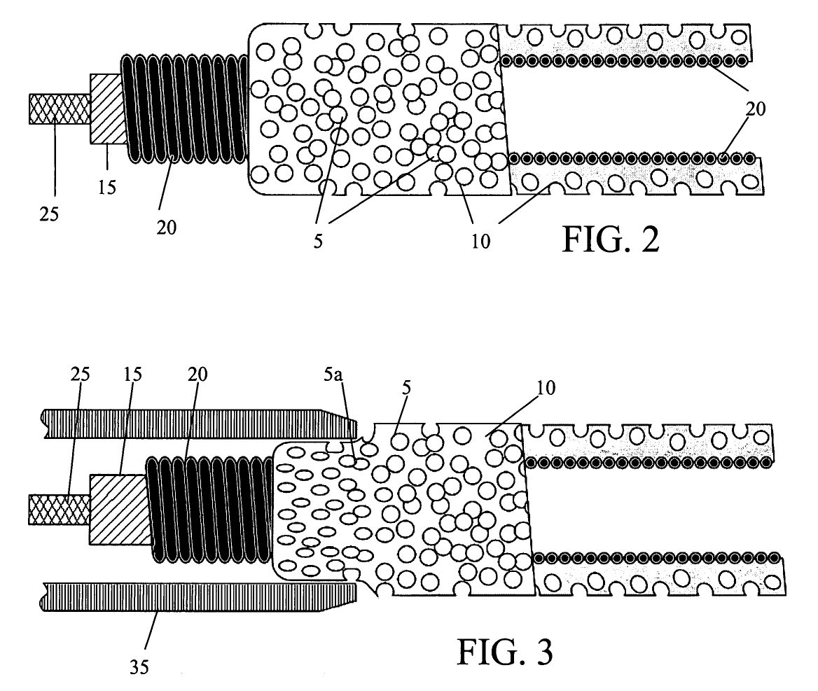

FIG. 2 shows the exemplary embodiment of FIG. 1 in combination with GDC-type vaso-occlusive coil (20). As shown in FIG. 2, the macroporous material may have a tubular shape that surrounds an inner vaso-occlusive member. Macroporous material may also extend into part or all of the lumen of the coil (20). Interconnected (co-continuous) pores (5) are depicted as overlapping circles. The macroporous component (10) can be permanently or temporarily attached in one or more locations to the coil (20) by any suitable attachment mechanism. Also shown is detachment junction (15) positioned on the proximal end of the coil (20) as well as pusher wire (25).

As noted above, the macroporous devices described herein may be compressible, for example for loading into a deployment catheter. FIG. 3 shows the exemplary embodiment of FIG. 2 as partially deployed from a deployment catheter (35). Within the catheter (35) the pores (5a) of macroporous component (10) are compressed. Upon deployment, the pores (5) expand to their relaxed state.

The devices described herein may also include one or more outer members covering the macroporous member. Thus, the macroporous member may surround and/or be surrounded by one or more structural members.

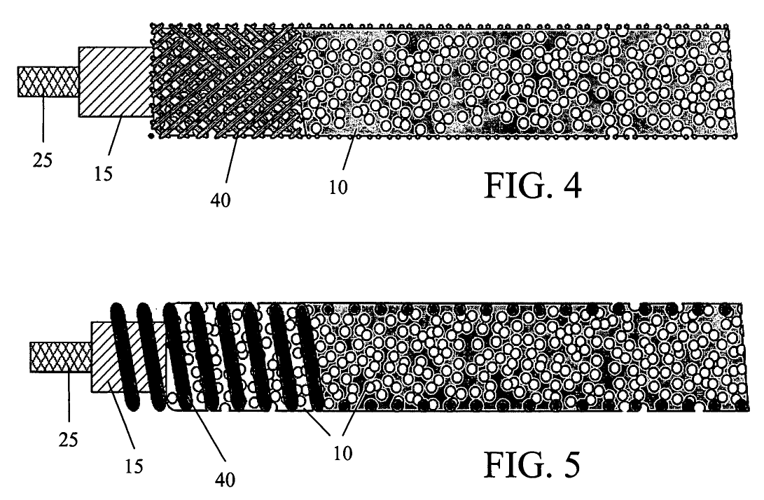

FIG. 4 shows another exemplary embodiment in which the macroporous component (10) is surrounded by an outer component (40). In this embodiment, outer component (40) comprises a tubular braid.

FIG. 5 shows another exemplary embodiment in which the macroporous component (10) is surrounded by an outer component (40), the outer component (40) having a coil shape in this embodiment.

The optional additional members (inner or outer) may assume a variety of structures. Thus, in addition to the coils and braids depicted in the Figures, other shapes are contemplated including, but not limited to, wires, knits, woven structures, tubes (e.g., perforated or slotted tubes), injection-molded devices and the like. See, e.g., U.S. Pat. No. 6,533,801 and International Patent Publication WO 02/096273.

Additionally, the additional structural member(s) (e.g., vaso-occlusive members) may be made of a variety of materials, including but not limited to metals, polymers and combinations thereof. In certain embodiments, the additional member(s) (e.g., braid, coil, etc.) comprises one or more metals or metal alloys, for example, Platinum Group metals, especially platinum, rhodium, palladium, rhenium, as well as tungsten, gold, silver, tantalum, stainless steel and alloys of these metals. Preferably, these elements comprise(s) a material that maintains its shape despite being subjected to high stress, for example, “super-elastic alloys” such as nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.” A shape memory polymer such as those described in International Publication WO 03/51444 may also be employed.

In certain preferred embodiments, the structural member comprises a vaso-occlusive platinum coil. The additional vaso-occlusive member may also change shape upon release from the restraining member, for example change from a constrained linear form to a relaxed, three-dimensional configuration upon deployment.

As shown in FIGS. 2 through 5, any of the devices described herein may further comprise a detachment junction (15), which is severable. The detachment junction (15) may be connected to a pusher element, such as a pusher wire (25). The detachment junction can be positioned anywhere on the device, for example at one or both ends of the structural element.

The severable junction(s) may be detached in a variety of ways, for example using an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof. Furthermore, the detachment mechanism may be hydraulic, for example the pusher wire may be cannulated, for example to allow for saline injection through the pusher wire to push off the coil.

The devices described herein may also comprise further additional components, such as co-solvents, plasticizers, coalescing solvents, bioactive agents, antimicrobial agents, porogens, antithrombogenic agents (e.g., heparin), antibiotics, pigments, radiopacifiers and/or ion conductors which may be coated using any suitable method or may be incorporated into the element(s) during production. See, e.g., co-owned U.S. patent application Ser. No. 10/745,911, U.S. Pat. No. 6,585,754 and WO 02/051460, incorporated by reference in their entireties herein. The bioactive materials can be coated onto the device (e.g., anticoagulants, growth factors, extracellular matrix components, living cells, DNA fragments, clotting stabilizers, or other materials intended to enhance or encourage wound healing) and/or can be placed in the vessel prior to, concurrently or after placement of one or more devices as described herein.

As noted elsewhere, the location of the device is preferably visible using fluoroscopy. A highly preferred method is to ensure that at least some of the elements (e.g., macroporous component and/or additional vaso-occlusive member) making up the device are provided with significant radio-visibility via the placement of a radio-opaque covering on these elements. A metallic coating of a metal having comparatively more visibility, during fluoroscopic use, than stainless steel is preferred. Such metals are well known but include gold and members of the Platinum Group described above.

One of more of the elements may also be secured to each other at one or more locations. For example, to the extent that various elements are thermoplastic, they may be melted or fused to other elements of the devices. Alternatively, they may be glued or otherwise fastened. Furthermore, the various elements may be secured to each other in one or more locations.

Methods of Use

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the vaso-occlusive device at the distal end, is advanced through the catheter.

Once the selected site has been reached, the vaso-occlusive device is extruded, for example by loading onto a pusher wire. Preferably, the vaso-occlusive device is loaded onto the pusher wire via a mechanically or electrolytically cleavable junction (e.g., a GDC-type junction that can be severed by application of heat, electrolysis, electrodynamic activation or other means). Additionally, the vaso-occlusive device can be designed to include multiple detachment points, as described in co-owned U.S. Pat. Nos. 6,623,493 and 6,533,801 and International Patent publication WO 02/45596. They are held in place by gravity, shape, size, volume, magnetic field or combinations thereof.

It will also be apparent that the operator can remove or reposition (distally or proximally) the device. For instance, the operator may choose to insert a device as described herein, before detachment, move the pusher wire to place the device in the desired location.

Modifications of the procedure and vaso-occlusive devices described above, and the methods of using them in keeping with this invention will be apparent to those having skill in this mechanical and surgical art. These variations are intended to be within the scope of the claims that follow.

What is claimed is:

1. A vaso-occlusive device comprising a macroporous material having a nominal pore size greater than about 30 microns, wherein at least 50 percent of the pores are interconnected with an adjacent pore.

2. The device of claim 1, wherein the pore size is between about 40 microns and about 400 microns.

3. The device of claim 1, wherein at least 80% of the pores are interconnected with an adjacent pore.

4. The device of claim 1, wherein the macroporous material comprises a polymer.

5. The device of claim 4, wherein the polymer is selected from the group consisting of silicones, polytetrafluoroethylene, polyesters, polyurethanes, proteins, hydrogel materials and combinations thereof.

6. The device of claim 1, further comprising a radioopaque material.

7. The device of claim 1, further comprising a structural element.

8. The device of claim 7, wherein the macroporous material at least partially surrounds, or is at least partially surrounded by, the structural element

9. The device of claim 8, wherein the macroporous material at least partially surrounds the structural element.

10. The device of claim 8, wherein the structural element at least partially surrounds the macroporous material.

11. The device of claim 7, comprising first and structural elements, wherein the macroporous material at least partially surrounds the first structural element and the second structural element at least partially surrounds the macroporous material.

12. The device of claim 7, wherein the macroporous material is attached to the structural element at one or more locations.

13. The device of claim 7, wherein the structural element comprises a metal.

14. The device of claim 13, wherein the metal is selected from the group consisting of nickel, titanium, platinum, gold, tungsten, iridium and alloys or combinations thereof.

15. The device of claim 14, wherein the metal is nitinol or platinum.

16. The device of claim 7, wherein the structural element comprises a coil, the coil comprising a metal selected from the group consisting of platinum, palladium, rhodium, gold, tungsten and alloys thereof.

17. The device of claim 7, wherein the structural element comprises a coil, the coil comprising a stainless steel or super-elastic metal alloy.

18. The device of claim 7, wherein the structural element comprises a tubular braid.

19. The device of claim 7, wherein the structural element comprises a biodegradable material.

20. The device of claim 7, wherein the structural element further comprises a detachment junction.

21. The device of claim 20, wherein the detachment junction comprises an electrolytically detachable end adapted to detach from a pusher by imposition of a current on the pusher.

22. The device of claim 1, further comprising bioactive component.

23. The device of claim 7, further comprising bioactive component.

24. A method of occluding a body cavity comprising introducing a vaso-occlusive device according to claim 1 into the body cavity.

25. The method of claim 23, wherein the body cavity is an aneurysm.
